(19) United States
(12) Patent Application Publication
(10) Pub. No.: US 2022/0054360 A1
(43) Pub. Date: Feb. 24, 2022

(54) CANNABIS-BASED SELF-EMULSIFYING PRODUCT

(71) Applicant: CannTrust Inc., Vaughan (CA)

(72) Inventors: Soheila HONARIKHEZRBEIGI, Toronto (CA); Jordan SILVER, Toronto (CA)

(73) Assignee: CannTrust Inc., Vaughan, ON (CA)

(21) Appl. No.: 17/311,528

(22) PCT Filed: Nov. 29, 2019
(86) PCT No.: PCT/CA2019/051709

§ 371 Date: Jun. 7, 2021

Related U.S. Application Data
(60) Provisional application No. 62/777,662, filed on Dec. 10, 2018.

(51) Int. Cl. A61J 3/07; A61K 31/05; A61K 36/185; A61K 31/352; A61K 47/14; A61K 9/50; A61K 47/26

(52) U.S. Cl. CPC A61J 3/074; A61K 31/05; A61K 36/185; A61K 9/5057; A61K 47/14; A61K 9/5031; A61K 47/26; A61K 31/352

(57) ABSTRACT

Cannabis-based self-emulsifying products such as self-emulsifying capsules are described herein together with methods for preparing such self-emulsifying products. In at least some embodiments, a self-emulsifying drug delivery system is described.

### TECHNICAL FIELD

The present disclosure relates to cannabis and, more particularly, to cannabis products and preparations such as cannabis-based self-emulsifying products, such as self-emulsifying capsules, suppositories, sublingual films and granulated powders.

### BACKGROUND

Cannabinoids, such as tetrahydrocannabinol (THC) and cannabidiol (CBD) are sometimes used for the treatment of various medical conditions. Since cannabinoids are hydrophobic, they may have a low bioavailability which presents a challenge for formulations. One strategy for solubilizing water-insoluble cannabinoids is oil in water nanoemulsion. However, such strategies may have dosing limitations and/or stability issues.

### BRIEF DESCRIPTION OF THE DRAWINGS

Reference will now be made, by way of example, to the accompanying drawings which show embodiments of the present application, and in which:

FIG. 1 is a flowchart of an example method of preparing a cannabis-based self-emulsifying product in accordance with the present disclosure;

FIG. 2 is a graph illustrating the distribution of droplet size for an emulsion of PEG-32 stearate as a SEDDS for a THC resin capsule dissolved in 100 ml of water;

FIG. 3 is a graph illustrating distribution of droplet size for a nanoemulsion of PEG-32 stearate and polysorbate 80 as a SNEDDS for a THC resin capsule after dissolving in water at 37° C.;

FIG. 4 is a graph illustrating distribution of droplet size for a nanoemulsion of PEG-32 stearate and polysorbate 80 with PEG 400 as a co-surfactant as a SNEDDS for a THC resin capsule after dissolving in water at 37° C.;

FIG. 5 is a graph illustrating the distribution of droplet size for a nanoemulsion of PEG-32 stearate and polysorbate 80 with PEG 400 as a co-surfactant as a SNEDDS for a THC (non-distilled) resin capsule after dissolving in an acid with a pH of 1.1 at 37° C.;

FIG. 6 is a graph illustrating distribution of droplet size for a nanoemulsion of PEG-32 stearate and polysorbate 80 with PEG 400 as a co-surfactant as a SNEDDS for a THC distilled resin capsule after dissolving in water at 37° C.;

FIG. 7 is a graph illustrating the distribution of droplet size for a nanoemulsion of PEG-32 stearate and polysorbate 80 with PEG 400 as a co-surfactant as a SNEDDS for a THC distilled resin capsule after dissolving in an acid with a pH of 1.1 at 37° C.;

FIG. 8 is a graph illustrating distribution of droplet size for a nanoemulsion of PEG-32 stearate and polysorbate 80 with PEG 400 as a co-surfactant as a SNEDDS for a CBD resin capsule after dissolving in water at 37° C.;

FIG. 9 is a graph illustrating the distribution of droplet size for a nanoemulsion of PEG-32 stearate and polysorbate 80 with PEG 400 as a co-surfactant as a SNEDDS for a CBD resin capsule after dissolving in an acid with a pH of 1.1 at 37° C.;

FIG. 10 is a graph illustrating the distribution of droplet size for a nanoemulsion of PEG-32 stearate and polysorbate 80 with PEG 400 as a co-surfactant as a SNEDDS for a CBD distilled resin capsule after dissolving in water at 37° C.;

FIG. 11 is a graph illustrating the distribution of droplet size for a nanoemulsion of PEG-32 stearate and polysorbate 80 with PEG 400 as a co-surfactant as a SNEDDS for a CBD distilled resin capsule after dissolving in an acid with a pH of 1.1 at 37° C.;

FIG. 12 is a graph illustrating the distribution of droplet size for a nanoemulsion of Lauroyl Polyoxyl-32 glycerides and polysorbate 80 with PEG 400 as a co-surfactant as a SNEDDS for a CBD resin capsule after dissolving in water at 37° C.; and

FIG. 13 is a graph illustrating the distribution of droplet size for a nanoemulsion of Gelucire 48/16 used at a low concentration and polysorbate 80 with PEG 400 as a co-surfactant as a SNEDDS for a CBD resin capsule after dissolving in water at 37° C.

Like reference numerals are used in the drawings to denote like elements and features.

### DETAILED DESCRIPTION OF EXAMPLE EMBODIMENTS

In an aspect a method of preparing a capsule may be described. The method may include preparing a mixture (which may also be referred to as a filling) that includes a cannabis-based preparation and a surfactant and adding the mixture/filling to a capsule. The surfactant may preferentially be PEG-32 stearate, but may also be polyoxyl stearates containing PEGs (Polyethylene glycols) of similar molecular sizes like stearoyl polyoxyl-32 glycerides (e.g., Gelucire 50/13), Lauroyl Polyoxyl-32 glycerides (e.g., Gelucire 44/14), Macrogol 15 Hydroxystearate (e.g., Kolliphor HS 15), Lauroyl polyoxyl-6 glycerides (e.g., Labrafil M 2130 CS), Caprylocaproyl Polyoxyl-8 glycerides (e.g., Labrasol). The cannabis-based preparation may include a cannabis resin or isolate. The cannabis resin or isolate may be selected from the group that includes: non-distilled CBD resin; non-distilled THC resin; THC distilled resin; and CBD distilled resin; CBD isolate; THC isolate.

The method may include, prior to preparing the mixture, melting the surfactant. Melting may include heating the surfactant at a temperature of between 50 to 65 degrees Celsius, but can also be accomplished at temperatures of at least 48 degrees Celsius. The heating may occur in a water bath or a double jacket melting tank.

The mixture may further include an antioxidant such as alpha tocopherol. The mixture may further include a carrier, such as an oil. For example, the carrier may may preferentially be MCT oil, but may be any kind of medium chain fatty acid or long chain fatty acid; for example: Glyceryl monooleate (e.g., Peceol) or Glyceryl monolinoleate (e.g., Maisine CC). The mixture may further include non-ionic surfactants with a hydrophilic-lipophilic balance (HLB) value more than 12, such as polysorbate 80. The mixture may further preferentially include PEG 400, or may include other low molecular weight co-surfactants such as PEG 300 or PEG 200.

The method may further include mixing the mixture until the mixture becomes clear yellow.

The capsule into which the mixture is added may be any kind of hard-shell or soft gel capsule like hydroxypropyl methylcellulose (HPMC) capsule or a gelatin capsule. The mixture may be heated during filling. For example, the mixture may be heated at between 45 and 50 degrees Celsius in some embodiments. The method may include, after adding the mixture to the capsule, allowing the mixture in the capsule to cool and sealing the capsule. Sealing the capsule may be performed without banding. Allowing the mixture in the capsule to cool may include allowing the mixture in the capsule to cool until the consistency of the mixture changes to a waxy semi solid, or liquid depending on the surfactants and carriers used.

In an aspect a capsule prepared according to a method described herein is described. In an aspect a capsule having a filling is described. The filling may include a cannabis-based preparation and a surfactant. The surfactant may preferentially be PEG-32 stearate, but may also be polyoxyl stearates with close PEG-sizes like stearoyl polyoxyl-32 glycerides (Gelucire 50/13), Gelucire 44/14, Kolliphor HS 15, Labrafil M 2130 CS, Labrasol. The cannabis-based preparation may include a cannabis resin. The cannabis resin is selected from the group that includes: non-distilled CBD resin; non-distilled THC resin; THC distilled resin; and CBD distilled resin. The filling may include alpha tocopherol (Vitamin E), Butylated Hydroxy Anisole (2(3)-t-Butyl-4 hydroxyanisole), Butylated Hydroxy Toluene (2,6-Di-tert-butyl-4-methylphenol) or another antioxidant. The filling may preferentially include MCT oil, but may also include LCT oil. The filling may include non-ionic surfactants with an HLB value more than 12, such as Polysorbate 80. The filling may preferentially include PEG 400 but it may also include other low molecular weight PEG such as PEG 300 and/or PEG 200. The filling may be a semisolid at room temperature, or may be a liquid depending on the surfactants and carrier oils used.

The capsule may include a capsule body and the capsule body may be any kind of hard-shell or soft gel capsule such as a hydroxypropyl methylcellulose (HPMC) capsule or a gelatin capsule. The capsule may not include banding.

Use of a capsule described herein, such as a capsule prepared according to a method described herein, for the treatment or amelioration of one or more symptoms or medical conditions are contemplated. The symptoms or medical conditions may include one or more of: inflammation, loss of appetite, nausea, vomiting, pain, chronic pain, muscle spasms, multiple sclerosis, glaucoma, AIDS, a neuropathic condition, cancer, acne, malnutrition, arthritis, chemotherapy induced nausea and vomiting, and/or a spinal cord injury.

Self-emulsifying products such as self-emulsifying capsules are described herein together with methods for preparing such self-emulsifying products. In at least some embodiments, a self-emulsifying drug delivery system (SEDDS) is described.

SEDDS are isotropic mixtures of drugs, lipids and surfactants. SEDDS may have one or more hydrophilic co-emulsifiers that form fine oil in water emulsions upon mild agitation in an aqueous medium. For example, self-emulsifying products may spontaneously emulsify in vivo. For example, the self-emulsifying products may emulsify in the gastrointestinal tract.

In at least some embodiments, the SEDDS may be self-nanoemulsifying drug delivery system (SNEDDS). Nano-emulsions may improve bioavailability by increasing the drug solubility, enhancing permeation across the intestinal membrane through a wide distribution in the gastrointestinal tract (due to the small droplet size) and decreasing the food effect (since foods may affect bioavailability). Nano-emulsions are defined as having a droplet size of up to 200 nm. In some embodiments, the SEDDS may not be a SNEDDS. For example, the droplet size may be larger than 200 nm. In some embodiments, the SEDDS may be a self-microemulsifying drug delivery system (SMEDDS). SMEDDS have a droplet size that is less than 250 nm.

The rapid emulsification of the self-emulsifying products in the gastrointestinal tract may provide improved oral bioavailability and/or a reproducible plasma concentration of a drug. Furthermore, the droplet size of the nanoemulsion would influence the extent of absorption of the drug when administered orally.

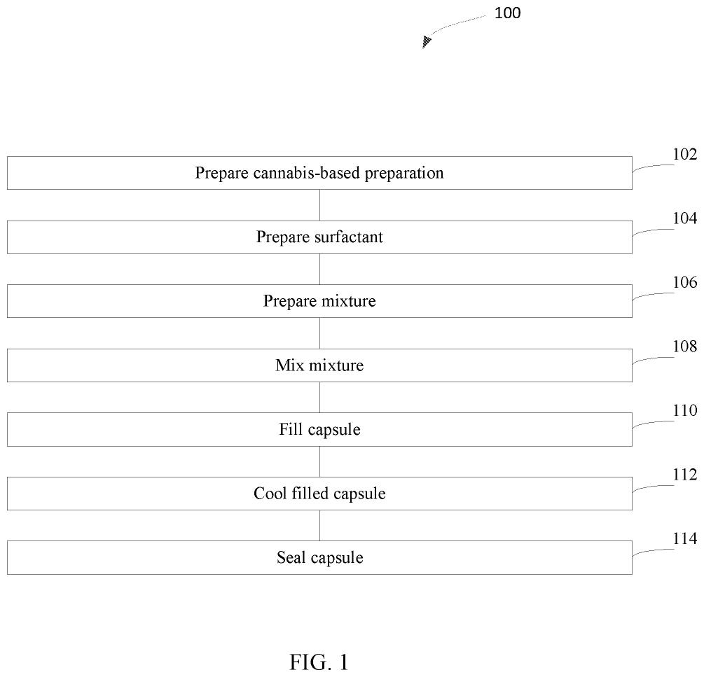

Reference is first made to FIG. 1, which illustrates a method 100 of preparing a cannabis-based self-emulsifying product such as a SEDDS.

At step 102, a cannabis-based preparation is prepared. The cannabis-based preparation may include, for example, a cannabis resin or cannabis isolate (such as CBD or THC isolate). For example, the cannabis-based preparation may include a cannabinoid resin or crystal CBD or THC. The cannabis resin may include one or more of tetrahydrocannabinol (THC) distilled resin, THC non-distilled resin, cannabidiol (CBD) distilled resin, CBD non-distilled resin or mixture of such resins. Other cannabinoids may be included in the cannabis resin instead of or in addition to those noted above. By way of example any one or a combination of THCV (tetrahydrocannabivarin), CBG (cannabigerol), CBDA (cannabidiolic acid), THCA (tetrahydrocannabinolic acid), CBN (cannabinol), or other cannabinoids may be included in the resin used at step 102.

Preparing the cannabis-based preparation at step 102 may include testing the cannabis-based preparation for potency and selecting an amount of the cannabis-based preparation based on the potency. That is, the cannabis-based preparation may be weighed based on the potency. For example, an amount of the cannabis-based preparation may be set aside for use in the subsequent steps of the method 100 and the amount may be based on the potency.

At step 104, a surfactant may be prepared. The surfactant may, for example, be a polyethylene glycol (PEG) based surfactant. The surfactant may preferentially be PEG-32 stearate, but may also be polyoxyl stearates containing PEGs (Polyethylene glycols) of similar molecular sizes like stearoyl polyoxyl-32 glycerides (e.g., Gelucire 50/13), Lauroyl Polyoxyl-32 glycerides (e.g., Gelucire 44/14), Macrogol 15 Hydroxystearate (e.g., Kolliphor HS 15), Lauroyl polyoxyl-6 glycerides (e.g., Labrafil M 2130 CS), Caprylocaproyl Polyoxyl-8 glycerides (e.g., Labrasol), which may act as a solubilizer, bioavailability enhancer and/or surfactant. In some implementations, the surfactant may be Gelucire™ 48/16.

While PEG-32 stearate has been found to work well, it is expected that other PEG stearates may be used instead of or in addition to PEG-32 stearate. For example, any one or a combination of the following may be useful as a substitute for or in combination with PEG-32: PEG-2, PEG-6, PEG-8, PEG-12, PEG-20, PEG-32, PEG-40, PEG-50, PEG-100, PEG-120, PEG-150.

At step 104, the surfactant may be prepared by measuring a desired amount of the surfactant and, in at least some embodiments, melting that amount of surfactant (which may be in pellet form at room temperature). The amount of PEG-32 stearate that is used will depend on the resin type. In at least some embodiments, the amount of PEG-32 stearate may be selected to maintain a ratio of PEG-32 stearate to MCT preferentially 2.5 to 6, but a SEDDS can also be formulated at a ratio of 2 to 10.

The melting may be performed in a water bath or, under agitation, in a double jacket melting tank, for example. The melting may be performed at a high temperature. For example, the melting may be performed at a temperature of at least 65 degrees Celsius, for example.

At step 106, a mixture may be prepared. The mixture includes the cannabis-based preparation prepared at step 102 and the surfactant prepared at step 104. The mixture may be prepared in a container which may, for example, be a container that previously included the cannabis based preparation or a container that previously included the surfactant. That is, the surfactant may be added to the cannabis-based preparation or the cannabis-based preparation may be added to the surfactant. Heat may be applied to the mixture at step 106 to prevent solidification of the mixture. For example, the heat may be applied using a water bath or, under agitation, in a double jacket melting tank, which may be the same equipment used at step 104.

In at least some embodiments, one or more other preparations may be added to the mixture at step 106. For example, in at least some embodiments an antioxidant may be added to the mixture. The antioxidant may be alpha tocopherol (which may also be referred to as Vitamin E), Butyalated Hydroxy Anisole (2(3)-t-Butyl-4 hydroxyanisole), Butyalated Hydroxy Toluene (2,6-Di-tert-butyl-4-methylphenol) or another antioxidant safe for oral use. The antioxidant may, for example, aid in preventing or inhibiting oxidation and/or degradation. This may, for example, enhance the stability and/or shelf life.

In some embodiments, a carrier oil such as medium chain triglyceride (MCT) or long chain triglyceride (LCT) oil may be added to the mixture. MCT or LCT may be used to provide the mixture with a consistency that makes it easier to use to fill a capsule.

In some embodiments, a further surfactant and/or emulsifier may be added to the mixture. For example, Polysorbate 80, such as Tween™ 80, may be added. Alternatively, in some embodiments, Polysorbate 60 may be preferentially used, but Polysorbate 60 to 85 can also be used.

As will be illustrated below, without the further surfactant (e.g., Polysorbate 80), a self-emulsifying product may be provided, such as a SEDDS, a SNEDDS, or a SMEDDS. As will be illustrated, however, polysorbate 80 may be used to provide a self-nanoemulsifying product, such as a SNEDDS. That is, the inclusion of Polysorbate 80 has been found to allow for a droplet size that is less than 200 nm and, therefore, may be considered nanoemulsifying. The polysorbate 80 may, for example, be approximately 1% W/W of the filling or 4% W/W of the oil phase.

In at least some embodiments, at step 106, a co-surfactant, such as a low-molecular-weight grade of polyethylene glycol, may be added to the mixture. For example, PEG 400 may be added at step 106. In other embodiments, PEG 200 or PEG 300 may be used instead of or in addition to PEG 400. The co-surfactant, such as PEG 400 may aid in creating smaller and/or more uniform nano-droplets.

The preparations that are added to the mixture at step 106 may be added in quantities that maintain a desired ratio of the ingredients. In at least some embodiments, vitamin E may be approximately 0.04% of the oil phase, the carrier oil, for example, MCT or LCT oil, may be ½-1/4.5 of PEG-32 stearate, polysorbate 80 may be approximately 4% of the oil phase and PEG 400 may be 10% of the surfactant mixture.

At step 108, the mixture may be stirred. The mixture may be stirred or otherwise mixed or agitated until the mixture becomes clear yellow (i.e., until it turns to a clear yellow liquid). In laboratory settings, such conditions have been observed after approximately five minutes of stirring. However, various factors may affect the period of stirring required such as, for example, the texture of the resin.

At step 110, the mixture may be used to fill one or more capsules. The capsules may be semisolid capsules. The capsules may, for example, be any kind of hard-shell or soft gel capsules such as hydroxypropylmethyl cellulose (HPMC) capsules or gelatin capsules. The capsules may be filled using a capsule filling machine. The capsules may be filled with a predetermined weight of mixture that achieves a desired dosage of CBD and/or THC per capsule. By way of example, for some capsules a fill weight of at least 0.2 g can be used to achieve a dosage of 10 mg THC/capsule in a number one (1) sized capsule. However, it will be appreciated that the fill weight required to achieve a desired dosage will vary based on numerous factors including, for example, the potency of the cannabis-based preparation and the ratio of the cannabis-based preparation to other components of the mixture.

At step 112, after filling, the method may include cooling the mixture in the capsule down so that the mixture solidifies. That is, the mixture may be allowed to cool so that it loses its liquid consistency. For example, the mixture may be a waxy semi solid at room temperature and it may be cooled until reaching such consistency. The cooling occurs quickly (e.g., it has been observed to occur in less than one minute in laboratory conditions). The cooling may, for example, continue until the mixture/filling reaches room temperature.

After cooling, at step 114, the capsule may be sealed. More particularly, a capsule cap may be placed over a capsule body (which is the portion of the capsule that was filled at step 110) to seal the capsule. Conveniently, in at least some embodiments, the texture of the mixture after cooling allows the capsule to be produced without the need for banding. Banding is often used to seal capsules filled with liquids. More specifically, banding seals a joint between a capsule cap and a capsule body in order to prevent leakage of liquid products. The PEG-32 stearate may contribute to the consistency of the mixture.

Conveniently, the mixture described above may have a polydispersity index (PDI) of less than 0.36 but it is preferably as low as 0.3.

Conveniently, the capsules described above may have a disintegration time of less than 20 minutes. Disintegration time is the time required for a dosage to break up into granules of a specified size (or smaller than a specified size) under specified conditions. That is, disintegration time is a measure of the breakdown of a dosage form. A lower disintegration time is generally considered desirable since higher disintegration times delay the onset of a drug.

Conveniently, results of dissolution testing has shown that more than 85% of the active substances in the capsules described herein may be dissolved after 60 minutes and dissolution of 95% of the active substances in the capsules described herein has even been observed after 60 minutes.

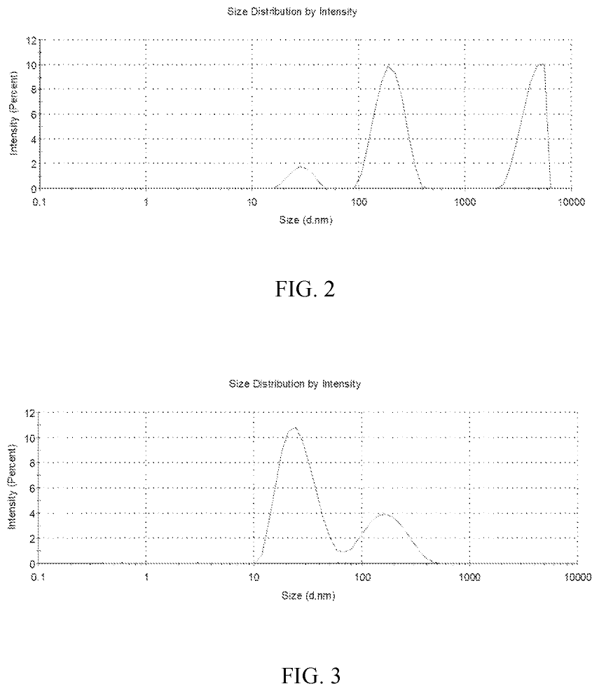

Referring now to FIG. 2, a graph illustrates the distribution of droplet size for an emulsion of PEG-32 stearate as a SEDDS for a THC resin capsule in water. That is the method 100 of FIG. 1 has been used to prepare the capsule. More specifically, non-distilled THC resin has been used at step 102 of the method and PEG-32 stearate has been melted at step 104. At step 106 of the method 100, the THC resin was combined with the PEG-32 stearate. Notably, neither Polysorbate 80 (or Polysorbate 60) or a co-surfactant such as PEG 400 (or PEG 200 or PEG 300) were added to the mixture. The mixture also included Vitamin E and MCT. As can be seen in FIG. 2, the emulsion has a small droplet size but not sufficiently small to be classified as a nanoemulsion. That is, the capsule produced without the Polysorbate 80 or PEG 400 may provide a SEDDS but not a SNEDDS with a droplet size of less than 200 nm.

Referring now to FIG. 3, a graph illustrating distribution of droplet size for a nanoemulsion of PEG-32 stearate as a SNEDDS for a THC resin capsule in water. That is, the method 100 of FIG. 1 has been used to prepare the capsule. The method used to prepare the capsule used in FIG. 3 is the same as the method used to prepare the capsule used in the previous example of FIG. 2 with the exception of the addition of Polysorbate 80. That is, non-distilled THC resin has been used at step 102 of the method and PEG-32 stearate has been melted at step 104. At step 106 of the method 100, the THC resin was combined with the PEG-32 stearate and also with Polysorbate 80. As with the example represented by FIG. 2, no co-surfactant, such as PEG 400 (or PEG 200 or PEG 300), was added to the mixture. The mixture also included Vitamin E and MCT. As can be seen in FIG. 3, the emulsion has a droplet size that is sufficiently small to be classified as a nanoemulsion. That is, the capsule produced with the Polysorbate 80 may provide a SNEDDS with a droplet size of less than 200 nm.

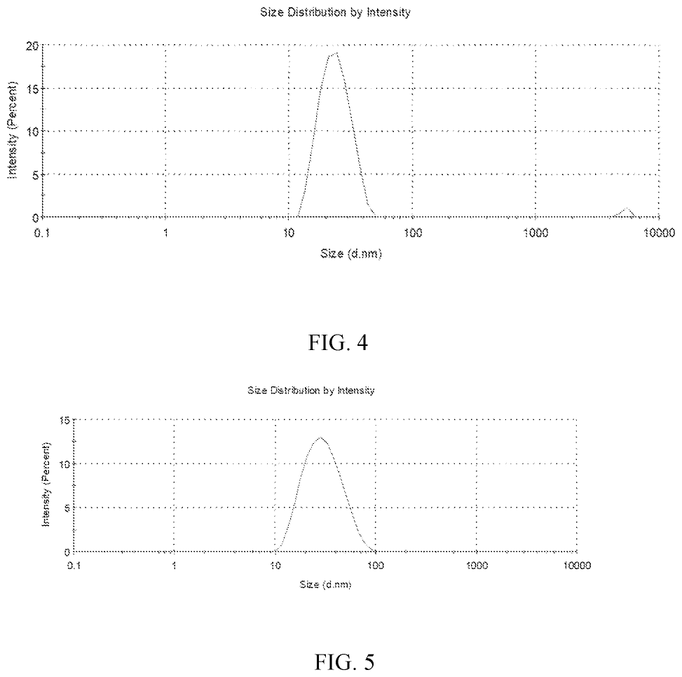

Referring now to FIG. 4, a further graph is illustrated. FIG. 4 illustrates the effect of a co-surfactant (PEG 400) on droplet size distribution. More specifically, FIG. 4 is a graph illustrating distribution of droplet size for a nanoemulsion of PEG-32 stearate with PEG 400 as a co-surfactant as a SNEDDS for a THC resin capsule in water. That is, the method 100 of FIG. 1 has been used to prepare the capsule. The method used to prepare the capsule used in FIG. 4 is the same as the method used to prepare the capsule used in the previous example of FIG. 3 with the exception of the addition of PEG 400 at step 106. That is, non-distilled THC resin has been used at step 102 of the method and PEG-32 stearate has been melted at step 104. At step 106 of the method 100, the THC resin was combined with the PEG-32 stearate and also with Polysorbate 80 and also with PEG 400. The mixture also included Vitamin E and MCT. As can be seen in FIG. 4, the emulsion has a droplet size that is sufficiently small to be classified as a nanoemulsion and the use of PEG 400 has improved the droplet size distribution. The capsule represented by FIG. 4 may provide a SNEDDS with a droplet size of less than 200 nm.

The capsule used for FIG. 4 also provides a nanoemulsion in acid. For example, FIG. 5 illustrates distribution of droplet size for a nanoemulsion of PEG-32 stearate with PEG 400 as a co-surfactant as a SNEDDS for a THC (non-distilled) resin capsule in an acid with a pH of 1.1. As illustrated in FIG. 5, the emulsion has a droplet size that is sufficiently small to be classified as a nanoemulsion. The acid-based emulsion may, for example, simulate gastric acid for an in vivo nanoemulsion.

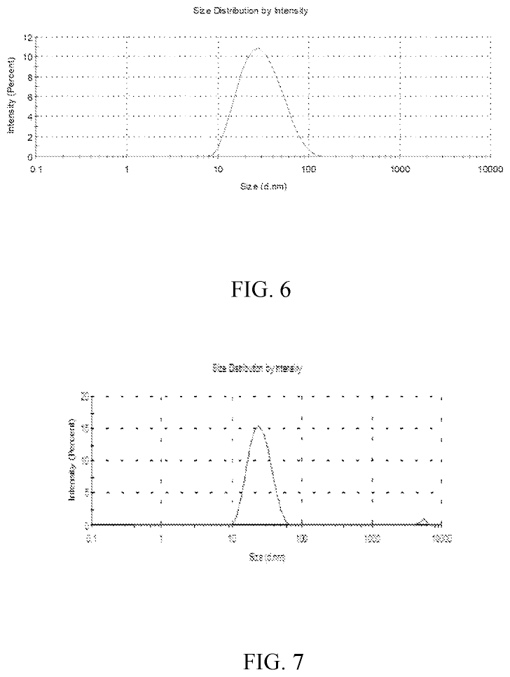

Referring now to FIG. 6, a further graph is illustrated. The capsule represented by FIG. 6 has been prepared using the same technique as the capsule represented by FIG. 4 with the exception of the resin. While the capsule of FIG. 4 used a non-distilled THC resin, the capsule of FIG. 6 used a distilled THC resin. Accordingly, FIG. 6 is a graph illustrating distribution of droplet size for a nanoemulsion of PEG-32 stearate with PEG 400 as a co-surfactant as a SNEDDS for a THC distilled resin capsule in water. As with the prior graphs, the method 100 of FIG. 1 has been used to prepare the capsule represented by FIG. 6. The method used to prepare the capsule used in FIG. 6 is the same as the method used to prepare the capsule used in the previous example of FIG. 4 except that distilled THC resin has been used at step 102 of the method. As before, PEG-32 stearate has been melted at step 104. At step 106 of the method 100, the distilled THC resin was combined with the PEG-32 stearate and also with Polysorbate 80 and also with PEG 400. The mixture also included Vitamin E and MCT. As can be seen in FIG. 6, the emulsion has a droplet size that is sufficiently small to be classified as a nanoemulsion. The capsule represented by FIG. 6 may provide a SNEDDS with a droplet size of less than 200 nm.

The capsule used for FIG. 6 also provides a nanoemulsion in acid. For example, FIG. 7 illustrates distribution of droplet size for a nanoemulsion of PEG-32 stearate with PEG 400 as a co-surfactant as a SNEDDS for a THC distilled resin capsule in an acid medium with a pH of 1.1. As illustrated in FIG. 7, the emulsion has a droplet size that is sufficiently small to be classified as a nanoemulsion. The acid-based emulsion may, for example, simulate gastric acid for an in vivo nanoemulsion.

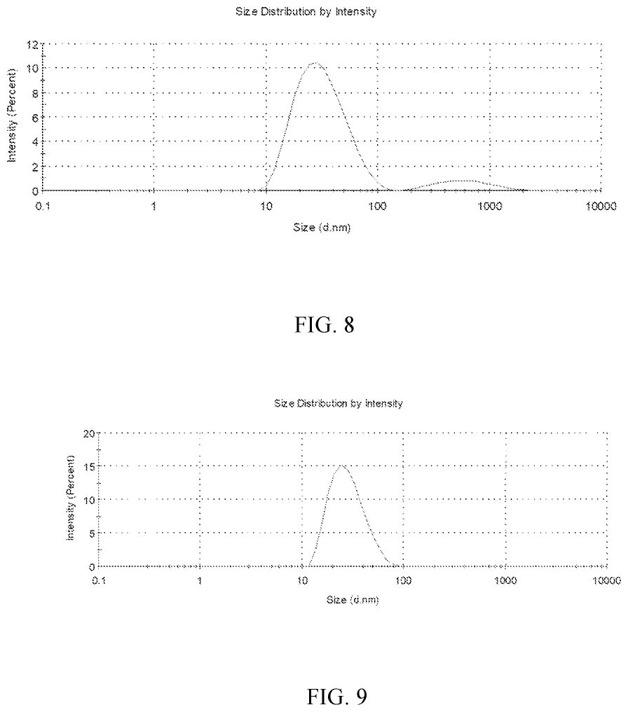

The method 100 of FIG. 1 has also been found to work well for CBD distilled and non-distilled resins. For example, referring now to FIG. 8, a further graph is illustrated. The capsule represented by FIG. 8 has been prepared using the same technique as the capsule represented by FIG. 4 with the exception of the resin. While the capsule of FIG. 4 used a non-distilled THC resin, the capsule of FIG. 8 used a non-distilled CBD resin. Accordingly, FIG. 8 is a graph illustrating distribution of droplet size for a nanoemulsion of PEG-32 stearate with PEG 400 as a co-surfactant as a SNEDDS for a CBD resin capsule in water. As with the prior graphs, the method 100 of FIG. 1 has been used to prepare the capsule represented by FIG. 8. The method used to prepare the capsule used in FIG. 8 is the same as the method used to prepare the capsule used in the previous example of FIG. 4 except that CBD resin has been used at step 102 of the method. As before, PEG-32 stearate has been melted at step 104. At step 106 of the method 100, the CBD resin was combined with the PEG-32 stearate and also with Polysorbate 80 and also with PEG 400. The mixture also included Vitamin E and MCT. As can be seen in FIG. 8, the emulsion has a droplet size that is sufficiently small to be classified as a nanoemulsion. The capsule represented by FIG. 8 may provide a SNEDDS.

The capsule used for FIG. 8 also provides a nanoemulsion in acid medium. For example, FIG. 9 illustrates distribution of droplet size for a nanoemulsion of PEG-32 stearate with PEG 400 as a co-surfactant as a SNEDDS for a CBD resin capsule in an acid medium with a pH of 1.1. As illustrated in FIG. 9, the emulsion has a droplet size that is sufficiently small to be classified as a nanoemulsion. The acid-based emulsion may, for example, simulate gastric acid for an in vivo nanoemulsion.

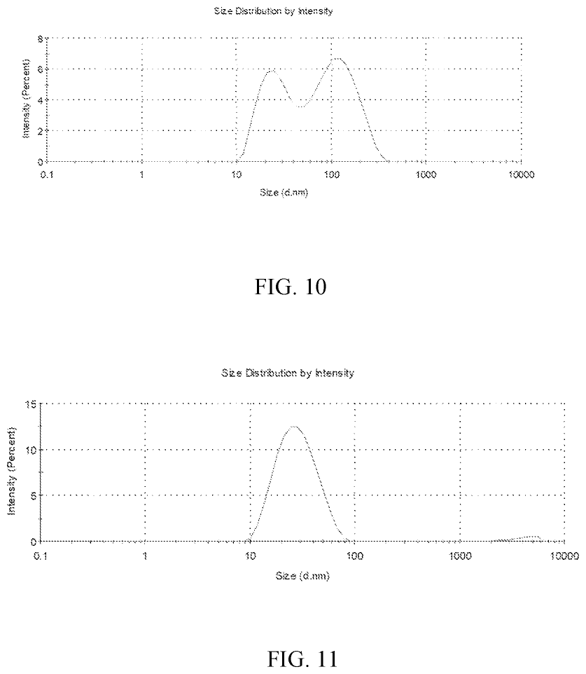

Referring now to FIG. 10, a further graph is illustrated. The capsule represented by FIG. 10 has been prepared using the same technique as the capsule represented by FIG. 8 with the exception of the resin. While the capsule of FIG. 8 used a non-distilled CBD resin, the capsule of FIG. 10 used a distilled CBD resin. Accordingly, FIG. 10 is a graph illustrating distribution of droplet size for a nanoemulsion of PEG-32 stearate with PEG 400 as a co-surfactant as a SNEDDS for a CBD distilled resin capsule in water. As with the prior graphs, the method 100 of FIG. 1 has been used to prepare the capsule represented by FIG. 10. The method used to prepare the capsule used in FIG. 10 is the same as the method used to prepare the capsule used in the previous example of FIG. 8 except that CBD distilled resin has been used at step 102 of the method. As before, PEG-32 stearate has been melted at step 104. At step 106 of the method 100, the CBD distilled resin was combined with the PEG-32 stearate and also with Polysorbate 80 and also with PEG 400. The mixture also included Vitamin E and MCT. As can be seen in FIG. 10, the emulsion has a droplet size that is sufficiently small to be classified as a nanoemulsion.

The capsule represented by FIG. 10 may provide a SNEDDS.

The capsule used for FIG. 10 also provides a nanoemulsion in an acid medium. For example, FIG. 11 illustrates distribution of droplet size for a nanoemulsion of PEG-32 stearate with PEG 400 as a co-surfactant as a SNEDDS for a CBD distilled resin capsule in an acid medium with a pH of 1.1. As illustrated in FIG. 11, the emulsion has a droplet size that is sufficiently small to be classified as a nanoemulsion. The acid-based emulsion may, for example, simulate gastric acid for an in vivo nanoemulsion.

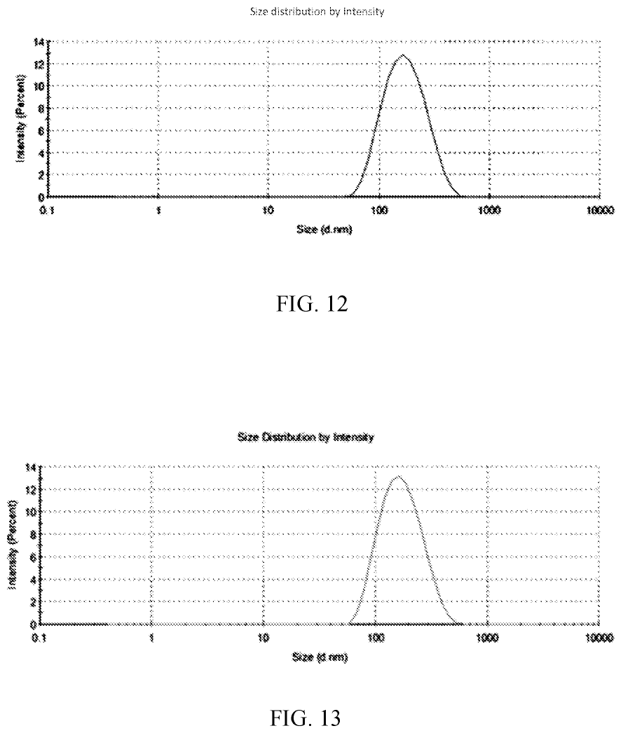

Referring now to FIG. 12, a further graph is illustrated. The capsule of FIG. 12 has been prepared using the same techniques as the capsule represented by FIG. 8 except that Lauroyl Polyoxyl-32 glycerides has been used as a surfactant instead of PEG-32 stearate. As illustrated in FIG. 12, the emulsion has a droplet size of about 200 nm and is therefore, approximately a nanoemulsion.

Referring now to FIG. 13, a further graph is illustrated. The capsule of FIG. 13 has been prepared using the same techniques as the capsule represented by FIG. 8 except that one half of the amount of PEG-32 stearate has been used to prepare the capsule of FIG. 13 as compared with the capsule of FIG. 8. As illustrated in FIG. 13, the emulsion has a droplet size of about 200 nm and is therefore, approximately a nanoemulsion.

The capsules produced according to the methods described herein may include a cannabis-based preparation, such as a cannabis resin. That is, a cannabis-based preparation, such as cannabis resin, may be used, together with other substances described herein as a filling within the capsules. The cannabis resin may be a cannabinoid resin of the type described above with reference to step 102 of the method 100. For example, the cannabis resin may include one or more of: non-distilled CBD resin, non-distilled THC resin, THC distilled resin, or CBD distilled resin. The capsules may also include a surfactant (i.e., the filling may include a surfactant). The surfactant may be of a type described above with reference to step 104 of the method 100. For example, The surfactant may preferentially be PEG-32 stearate, but may also be polyoxyl stearates containing PEGs (Polyethylene glycols) of similar molecular sizes like stearoyl polyoxyl-32 glycerides (e.g., Gelucire 50/13), Lauroyl Polyoxyl-32 glycerides (e.g., Gelucire 44/14), Macrogol 15 Hydroxystearate (e.g., Kolliphor HS 15), Lauroyl polyoxyl-6 glycerides (e.g., Labrafil M 2130 CS), Caprylocaproyl Polyoxyl-8 glycerides (e.g., Labrasol). The filling of the capsules may also include alpha tocopherol. The filling of the capsules may also include MCT oil or LCT oil. The filling of the capsules may also include non-ionic surfactants with HLB value more than 12, preferentially Polysorbate 80. The filling of the capsules may include low molecular weight PEG such as PEG 400, PEG 300 and/or PEG 200.

The filling of the capsule may be a semisolid at room temperature. For example, the filling may be a waxy semisolid. In some embodiments, the filling may be a liquid and the consistency will depend on the surfactant(s) and the carrier oil(s) used.

The capsules may be any kind of hard-shell or soft gel capsule such as HPMC capsule or a gelatin capsule. The capsule may not include banding since the filling is a semisolid at room temperature.

The cannabis-based products described herein, such as the self-emulsifying capsules, may be used by a human or animal. For example, the cannabis-based products may be ingested (i.e., used orally). The cannabis-based products may be administered, for example, for medicinal benefits.

The cannabis-based products described herein may be used, for example, to treat a variety of medical conditions. For example, the cannabis-based products described herein, such as the self-emulsifying capsules, may be used for the treatment or amelioration of symptoms of medical conditions. Such symptoms may include any one or a combination of inflammation, lack of appetite, nausea, vomiting, chemotherapy induced nausea and vomiting, pain including chronic pain, or muscle spasms. For example, the cannabis based products described herein may be used as part of a treatment plan (including to manage symptoms) for conditions such as multiple sclerosis, glaucoma, AIDS, neuropathic conditions, cancer, acne, diseases of malnutrition, arthritis, or spinal cord injury. It can be appreciated that cannabis based products can be used for treatment of other symptoms or other conditions. Accordingly, the self-emulsifying capsules may be used for the treatment of any one or more medical conditions or systems, such as those described above. For example, the self-emulsifying capsules may be ingested by a patient suffering from such a symptom or condition.

In a variation of the above-described method, the filling/mixture that is described above may not be included in a capsule. Instead, the filling/mixture may be consumed directly by a user. In some embodiments, the filling/mixture may be processed into a small form, such as a powder. This may be done, for example, by grinding or otherwise breaking down the solidified form of the filling/mixture or the small form could be created through pouring of the liquid or otherwise separating the liquid into small parts before cooling. The filling or mixture may, for example, be added to a beverage or a food product for consumption.

The various embodiments presented above are merely examples. Variations of the innovations described herein will be apparent to persons of ordinary skill in the art, such variations being within the intended scope of the present application. In particular, features from one or more of the above-described example embodiments may be selected to create alternative example embodiments including a sub-combination of features which may not be explicitly described above. In addition, features from one or more of the above-described example embodiments may be selected and combined to create alternative example embodiments including a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A method of preparing a capsule, the method comprising:
preparing a mixture that includes a cannabis-based preparation, at least one surfactant and a co-surfactant; and
adding the mixture to a capsule.

2. The method of claim 1, wherein the mixture includes both Gelucire 48/16 and Polysorbate 80 as surfactants.

3. The method of claim 2, wherein the co-surfactant is PEG 400.

4. The method of claim 1, wherein the surfactant is PEG-32 stearate.

5. The method of claim 1, wherein the cannabis-based preparation includes a cannabis resin.

6. The method of claim 5 wherein the cannabis resin is selected from the group that includes:
non-distilled CBD resin;
non-distilled THC resin;
THC distilled resin; and
CBD distilled resin.

7. The method of claim 1, further comprising, prior to preparing the mixture, melting the surfactant.

8. The method of claim 7, wherein melting comprises heating the surfactant at a temperature of at least 48 degrees Celsius.

9. The method of claim 8, wherein heating comprises heating in a water bath.

10. The method of claim 8, wherein heating comprises heating in a double jacket melting tank.

11. The method of claim 1, wherein the mixture further includes alpha tocopherol.

12. The method of claim 1, wherein the mixture further includes MCT oil.

13. The method of claim 1, wherein the at least one surfactant includes Polysorbate 80.

14. The method of claim 1, wherein the co-surfactant includes PEG 400.

15. The method of claim 1, wherein the co-surfactant includes PEG 300.

16. The method of claim 1, wherein the co-surfactant includes PEG 200.

17. The method of claim 1, further comprising, mixing the mixture until the mixture becomes clear yellow.

18. The method of claim 1, wherein the capsule is a soft gel capsule.

19. The method of claim 1, wherein the capsule is a gelatin capsule.

20. The method of claim 1, wherein the capsule is a HPMC capsule.

21.-57. (canceled)
